(19) United States
(12) Patent Application Publication
(10) Pub. No.: US 2015/0126896 A1
(43) Pub. Date: May 7, 2015

(54) HUMAN BODY THERMAL MEASUREMENT DEVICE, A METHOD FOR MEASURING HUMAN BODY TEMPERATURE, AND A NON-TRANSITORY COMPUTER READABLE STORAGE MEDIUM

(71) Applicant: Raed H. AlHazme, Florham Park, NJ (US)

(72) Inventor: Raed H. AlHazme, Florham Park, NJ (US)

(21) Appl. No.: 14/073,294

(22) Filed: Nov. 6, 2013

(51) Int. Cl. A61B 5/01

(52) U.S. Cl. CPC A61B 5/01
USPC 600/549

(57) ABSTRACT

A human body thermal measurement device including a first sensor configured to measure a first temperature of a core part of the human body, a second sensor configured to measure a second temperature of a peripheral part of the human body, a third sensor configured to measure an ambient temperature surrounding the human body, a memory configured to store information on the first temperature, the second temperature and the ambient temperature, circuitry configured to calculate a difference between the first temperature and the second temperature, and change a first predetermined range according to the ambient temperature, and a user interface configured to output first alert when the difference is determined to be not within the first predetermined range by the circuitry.

### GRANT OF NON-EXCLUSIVE RIGHT

This application was prepared with financial support from the Saudia Arabian Cultural Mission, and in consideration therefore the present inventor(s) has granted The Kingdom of Saudi Arabia a non-exclusive right to practice the present invention.

### BACKGROUND

1. Field of the Disclosure

From a medical perspective, it is important to know the human body thermal status. The present disclosure relates to a human body thermal measurement device that can identify the status and/or reaction of the human body in relation to body thermal condition.

### SUMMARY

The foregoing paragraphs have been provided by way of general introduction, and are not intended to limit the scope of the following claims. The described embodiments, together with further advantages, will be best understood by reference to the following detailed description taken in conjunction with the accompanying drawings.

Among other things, the present disclosure provides a human body thermal measurement device that includes a first sensor configured to measure a first temperature of a core part of the human body, a second sensor configured to measure a second temperature of a peripheral part of the human body, a third sensor configured to measure an ambient temperature surrounding the human body, a memory configured to store information on the first temperature, the second temperature and the ambient temperature, circuitry configured to calculate a difference between the first temperature and the second temperature, change a first predetermined range according to the ambient temperature, determine if the difference is within the first predetermined range or not, determine if the first temperature is within a second predetermined range or not, determine if the second temperature is within a third predetermined range or not, determine if a change of the first temperature in time is within a fourth predetermine range or not, and determine if a change of the second temperature in time is within a fifth predetermined range or not, and a user interface configured to output first alert when the difference is determined to be not within the first predetermined range by the circuitry, and output second alert when either the first temperature is determined to be not within the second predetermined range, the second temperature is determined to be not within the third predetermined range, the change of the first temperature in time is determined to be not within the fourth predetermined range, or the change of the second temperature in time is determined to be not within the fifth predetermined range by the circuitry.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1(a) is a first exemplary human body thermal status;

FIG. 1(b) is a second exemplary human body thermal status;

FIG. 1(c) is a third exemplary human body thermal status;

FIG. 2 is an exemplary system for measuring human body temperature according to one embodiment;

FIG. 3 is an exemplary block diagram of a human body thermal measurement device according to one embodiment;

FIG. 4 is an exemplary flowchart of the human body thermal measurement device according to one embodiment;

FIG. 5 is an exemplary user interface of the human body thermal measurement device according to one embodiment;

FIG. 6 is an exemplary user interface of the human body thermal measurement device according to another embodiment;

FIG. 7 is an exemplary system for measuring human body temperature according to one embodiment;

FIG. 8 is an exemplary flow chart of a system for measuring human body temperature according to one embodiment; and

FIG. 9 is an exemplary system for measuring human body temperature according to another embodiment.

### DETAILED DESCRIPTION OF THE EMBODIMENTS

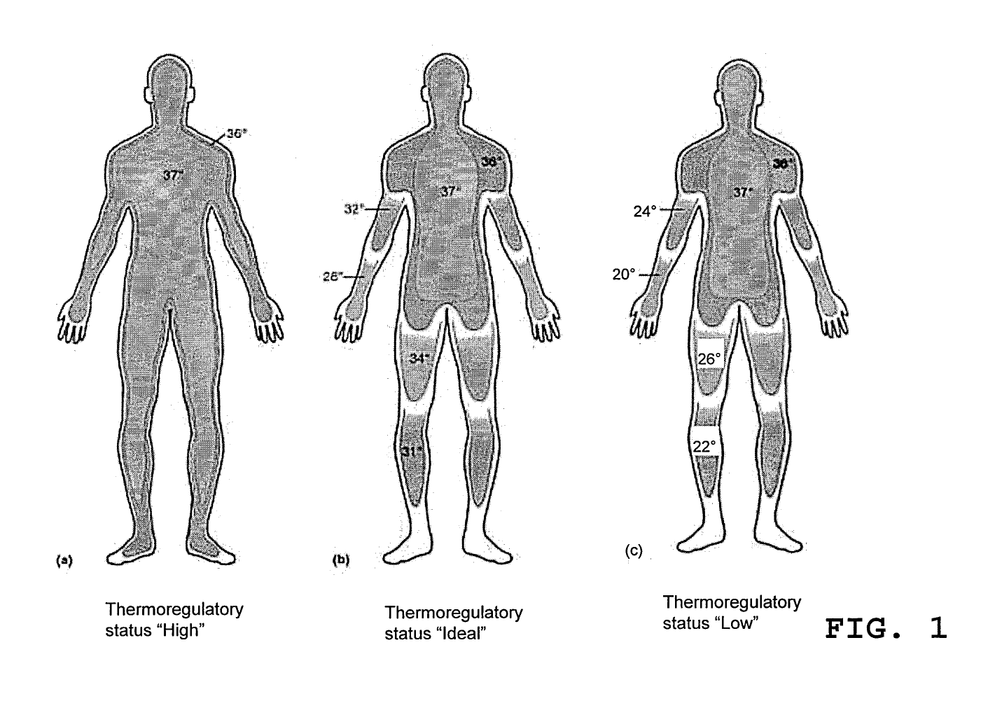

Referring now to the drawings, wherein like reference numerals designate identical or corresponding parts throughout the several views, FIG. 1(a) is a first exemplary human body thermal status, FIG. 1(b) is a second exemplary human body thermal status, and FIG. 1(c) is a third exemplary human body thermal status. Controlled by a part of the brain called the hypothalamus, the human body has a mechanism medically known as “thermoregulatory system”, in which human body temperature is monitored and adjusted when it is not in an ideal state. The ideal human body core temperature state where no temperature adjustment is necessary is called “normothermia” or “euthermia”, which typically falls between 36.5° C. (97.7° F.) and 37.5° C. (99.5° F.). The thermoregulatory system acts when excess heat exists in the core part of the body. In this case, as shown in FIG. 1(a), the thermoregulatory system causes peripheral vasodilation in order to dissipate the excessive heat in the core part (i.e., thermoregulatory status is “high”). This causes the temperature of peripheral part of the body (peripheral temperature) to be closer to the core part. FIG. 1(b) shows an exemplary ideal thermoregulatory status called “normothermia” or “euthermia”. When the core temperature falls below the “normothermia” or “euthermia” limit, the human body also acts to readjust the temperature and to prevent hypothermia. The first action taken by the thermoregulatory system is peripheral vasoconstriction, which decreases the flow of blood in the area. As shown in FIG. 1(c), this causes the peripheral part temperature to be much lower than the core part compared with the body's normal condition (i.e., thermoregulatory status is “low”).

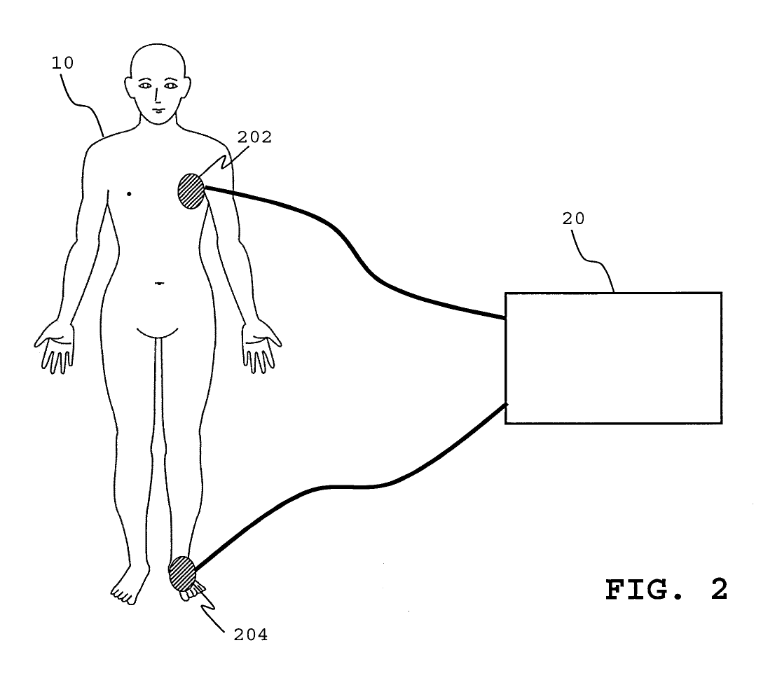

FIG. 2 is an exemplary system for measuring human body temperature according to one embodiment. A human body thermal measurement device 20 can identify the status and reaction of the human body 10 in relation to the body thermal condition. For example, the human body thermal measurement device 20 identifies if the human body 10 is in ideal temperature state or if the body is in the process of readjusting the temperature of some parts of the body because or hypo or hyperthermia. In order to identify the body thermal condition or the status of the thermoregulatory system, the human body thermal measurement device 20 monitors the core and peripheral temperatures of the human body 10 and constantly calculates the difference between the core temperature and the peripheral temperature. The core and peripheral temperatures of the human body 10 are measured by temperature sensors 202 and 204. Core temperature is a temperature of core part, specifically in deep structures of the human body 10, such as, organs, mouth, axilla, or brain Nonetheless, it can be relatively measured from the skin of the body torso. Peripheral temperature is a temperature of peripheral parts of the human body, such as arms and legs.

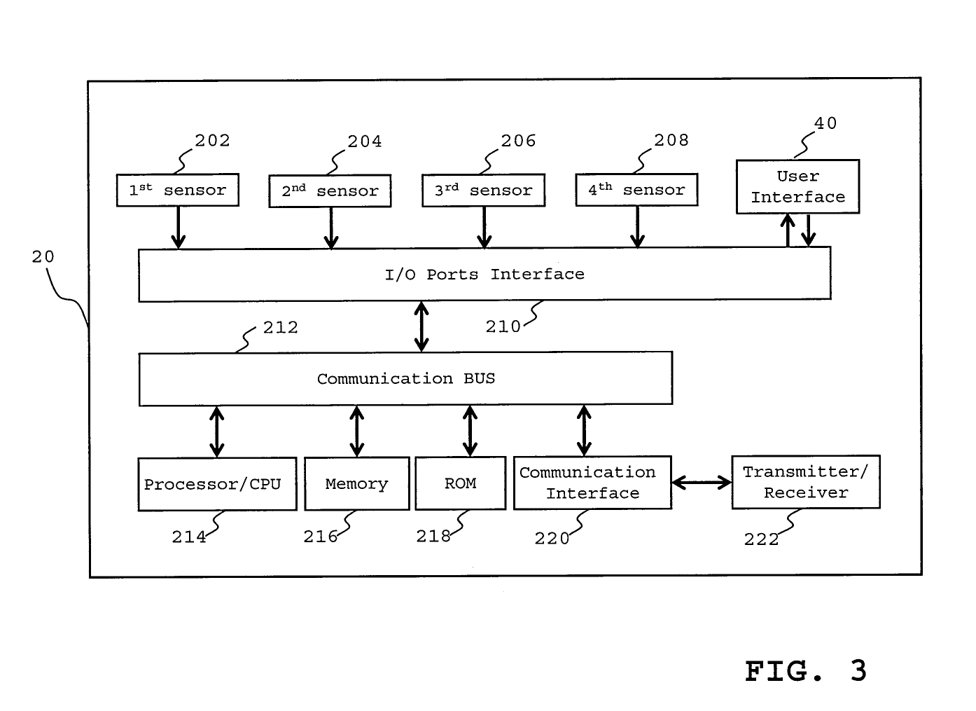

FIG. 3 is an exemplary block diagram of a human body thermal measurement device according to one embodiment. The human body thermal measurement device 20 includes a 1^{st} sensor 202 that measures core temperature of the human body 10 and 2^{nd} sensor 204 that measures the peripheral temperature of the human body 10. The 1^{st} sensor 202 and the 2^{nd} sensor 204 may be various kinds of thermometers, such as, thermistor type thermometer, radiometric thermometer, mercury-in-glass thermometer or bi-metallic thermometer. The 1^{st} sensor 202 and the 2^{nd} sensor 204 may be separate sensors or they can be implemented as one sensor covering both core and peripheral parts of the human body. The 1^{st} sensor 202 and/or the 2^{nd} sensor 204 may be implemented in clothes so that the core and peripheral part of the human body 10 is measured by the 1^{st} sensor 202 and the 2^{nd} sensor 204 easily just by wearing the clothes. 3^{rd} sensor 206 measures ambient temperature of the human body 10. For example, the 3^{rd} sensor 206 is a thermometer that measures a temperature of the room where the examinee 10 is. The human body thermal measurement device 20 may further include a 4^{th} sensor 208 to detect if the 1^{st} sensor 202 and/or the 2^{nd} sensor 204 contacts with the human body 10. The 4^{th} sensor is, such as, a thermocouple sensor, a thermistor sensor, a resistance sensor or a galvanic skin response sensor that is implemented to the part of the 1^{st} sensor 202 and/or the 2^{nd} sensor 204 to detect if the 1^{st} sensor 202 and/or the 2^{nd} sensor 204 are firmly attached to the human body 10.

User interface 40 is such as a display (e.g., LCD), an indicator (e.g., LED lamp) or a speaker that alert the measurement result. The 1^{st} sensor 202, 2^{nd} sensor 204, 3^{rd} sensor 206 and the user interface 40 are connected to I/O ports interface 210 and exchange data with each other. The I/O ports interface 210 may include logic to interpret the device address generated by the processor/CPU 214. The I/O ports interface 210 may also include a hand-shaking logic so that the processor/CPU 214 can communicate with the sensors 202, 204 and 206, and user interface 40 through the I/O ports interface 210. The I/O ports interface 210 is also connected to communication BUS 212. Communication BUS 212 is also connected to processor/CPU 214, memory 216, ROM 218 and communication interface 220. Communication BUS 212 stores information and instructions to be executed by the processor/CPU 214 and manages the signal transaction between each component in the human body thermal measurement device 20. The communication BUS 212 may include a data bus to carry information, an address bus to determine where the information should be sent and a control bus to determine its operation.

Processor/CPU 214 executes one or more sequences of one or more instructions contained in a memory, such as memory 216. Such instructions may be read into the memory 216 from another computer readable medium, such as a hard disk or removable media drive. One or more processors in a multi-processing arrangement may also be employed to execute the sequences of instructions contained in memory 216. In alternative embodiments, hard-wired circuitry may be used in place of or in combination with software instructions. Thus, embodiments are not limited to any specific combination of hardware circuitry and software.

As stated above, the human body thermal measurement device 20 includes at least one computer readable medium or memory, such as memory 216, for holding instructions programmed according to the teachings of the present disclosure and for containing data structures, tables, records, or other data described herein. Examples of non-transitory storage device are compact discs, hard disks, floppy disks, tape, magneto-optical disks, PROMs (EPROM, EEPROM, flash EPROM), DRAM, SRAM, SDRAM, or any other magnetic medium, compact discs (e.g., CD-ROM), or any other optical medium, punch cards, paper tape, or other physical medium with patterns of holes, a carrier wave, or any other medium from which a computer can read.

Stored on any one or on a combination of computer readable medium, the present disclosure includes software for controlling the human body thermal measurement device 20. Such software may include, but is not limited to, device drivers, operating systems, development tools, and applications software. Such computer readable medium further includes the computer program product of the present disclosure for performing all or a portion (if processing is distributed) of the processing performed in implementing the disclosure.

The computer code devices of the present disclosure may be any interpretable or executable code mechanism, including but not limited to scripts, interpretable programs, dynamic link libraries (DLLs), Java classes, and complete executable programs. Moreover, parts of the processing of the present disclosure may be distributed for better performance, reliability, and/or cost.

The term “computer readable medium” as used herein refers to any non-transitory or transitory medium that participates in providing instructions to the processor/CPU 214 for execution. A computer readable medium may take many forms, including but not limited to, non-volatile media, volatile media, and transmission media. Non-volatile media includes, for example, optical magnetic disks, and magneto-optical disks, such as the hard disk or the removable media drive. Volatile media includes dynamic memory, such as the memory 216. Transmission media includes coaxial cables, copper wire and fiber optics, including the wires that make up the communication bus 212. Transmission media may also take the form of acoustic or light waves, such as those generated during radio wave and infrared data communications.

Various forms of computer readable media may be involved in carrying out one or more sequences of one or more instructions to processor/CPU 214 for execution. For example, the instructions may initially be carried on a magnetic disk of a remote computer. The remote computer can load the instructions for implementing all or a portion of the present disclosure remotely into a dynamic memory and send the instructions over a telephone line using a modem. A modem local to the human body thermal measurement device 20 may receive the data on the phone line and use an infrared transmitter to convert the data to an infrared signal. An infrared detector coupled to the communication bus 212 can receive the data carried in the infrared signal and place the data on the communication bus 212. The communication bus 212 carries data to the memory 216, from which the processor/CPU 214 retrieves and executes the instructions. The instructions received by the memory 216 may optionally be stored on storage device either before or after execution by processor/CPU 214.

Memory 216 is any non-transitory storage device such as compact discs, hard disks, floppy disks, tape, magneto-optical disks, PROMs (EPROM, EEPROM, flash EPROM), random access memory (RAM), DRAM, SRAM, SDRAM, or any other magnetic medium, compact discs (e.g., CD-ROM), or any other optical medium, punch cards, paper tape, or other physical medium with patterns of holes, a carrier wave, or any other medium from which a computer can read and coupled to the communication bus 212 for storing information and instructions by the processor/CPU 214. In addition, the memory 216 may be used for storing temporary variables or other intermediate information during the execution of instructions by the processor/CPU 214.

The human body thermal measurement device 20 further includes a read only memory (ROM) 218 or other static storage device (e.g., programmable ROM (PROM), erasable PROM (EPROM), and electrically erasable PRM (EEPROM)) coupled to the communication bus 212 for storing static information and instructions for the processor/CPU 214.

The human body thermal measurement device 20 may also include a communication interface 220 coupled to the communication BUS 212. The communication interface 220 provides a two-way data communication coupling to a network link that is connected to, for example, wired communication network (e.g., LAN) or wireless communication network (e.g., cellular networks or wireless LAN) connected to the internet 90. In any such implementation, the communication interface 220 sends and/or receives electrical, electromagnetic or optical signals that carry digital data streams representing various types of information. The communication interface 220 may be further connected to a transmitter/receiver 222 including a transmitter and a receiver.

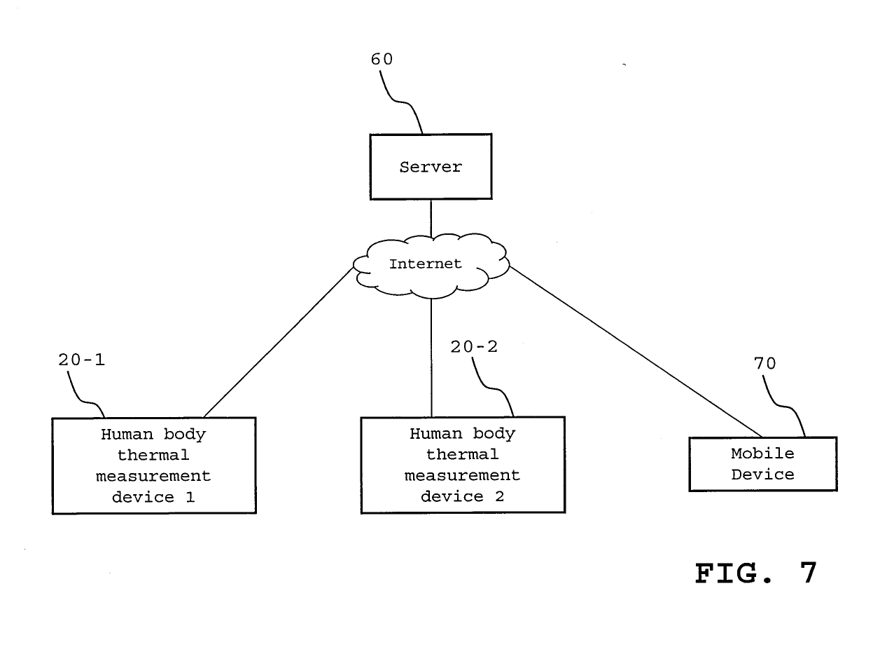

The network link typically provides data communication through one or more networks to other data devices. For example, the network link may provide a connection to the server 60, another human body thermal measurement device 20-2, or mobile device 70 through the internet (see FIG. 7). The human body thermal measurement device 20 may transmit and/or receive data, including program code, through the network(s), the network link, the transmitter/receiver 222 and the communication interface 220. The processor/CPU 214 may control the transmitter/receiver 222 to transmit information obtained by the sensors 202, 204 and 206, or information input to the user interface 40 to the server 60, another human body thermal measurement device 20-2, or mobile device 70 through the internet. The processor/CPU 214 may control the transmitter/receiver 222 to receive information from the server 60, another human body thermal measurement device 20-2, or mobile device 70 through the internet and control the user interface 40 to output the received information.

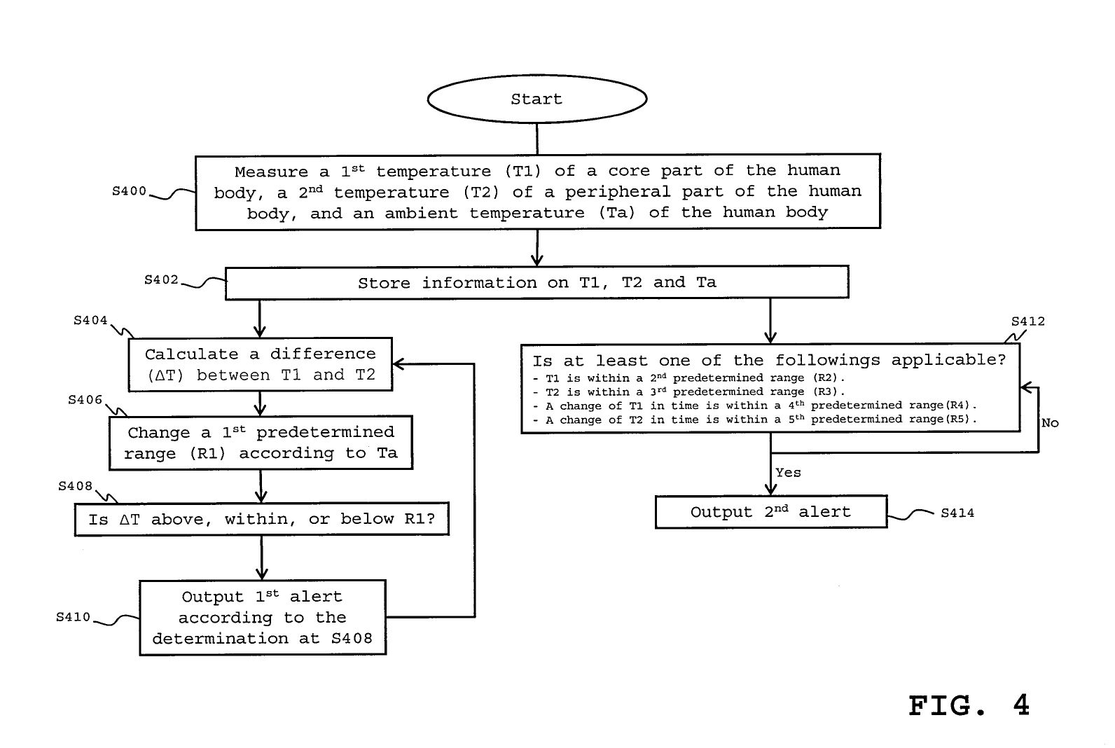

FIG. 4 is an exemplary flowchart of the human body thermal measurement device according to one embodiment. At S400, the 1^{st} sensor 202 measures a 1^{st} temperature (T1) of a core part of the human body 10, the 2^{nd} sensor 204 measures a 2^{nd} temperature (T2) of a peripheral part of the human body 10, and 3^{rd} sensor 206 measures an ambient temperature (Ta) of the human body 10. Then at S402, information on the T1, T2 and Ta measured by the 1^{st} sensor 202, the 2^{nd} sensor 204 and the 3^{rd} sensor 206 is stored in the memory 216. For example the information on the T1, T2 and Ta is store periodically (e.g., every 5 seconds) to reduce the amount of data in the memory 216, and/or the data can be erased after predetermined time (e.g., 1 hour) or after the data amount in the memory 216 exceeds predetermined amount (e.g., 1 Gb).

Then at S404, the processor/CPU 214 calculates a difference (ΔT) between T1 and T2. In general, core temperature (T1) is higher than peripheral temperature (T2) and thus the difference (ΔT) is expressed by {T1-T2}, or the processor/CPU 214 may calculate an absolute value of the difference (ΔT). The processor/CPU 214 may control the user interface 40 to output alert when T2 is higher than T1 as there might be some trouble (e.g., the 1^{st} sensor is not attached to the human body 10).

At 406, the processor/CPU 214 changes a 1^{st} predetermined range (R1) stored in the memory 216 according to the ambient temperature (Ta). For example, in the beginning the 1^{st} predetermined range (R1) is set to be 10-20° F. Then when the ambient temperature (Ta) becomes higher than a 1^{st} predetermined temperature (e.g., 95° F.), the 1^{st} predetermined range (R1) is changed to a range with smaller value (e.g., 1-10° F.), and/or when the ambient temperature (Ta) becomes lower than a 2^{nd} predetermined temperature (e.g., 50° F.), the 1^{st} predetermined range (R1) is changed to a range with higher value (e.g., 20-35° F.). This adjustment on the 1^{st} predetermined range (R1) is done to reduce the effect by the ambient temperature change because the peripheral temperature is easy to be affected by the ambient temperature compared with the core temperature. Therefore, for example, even though the difference (ΔT) of 5° F. under the ambient temperature of 65° F. is not an ideal status, it may be ideal under the ambient temperature of 95° F.

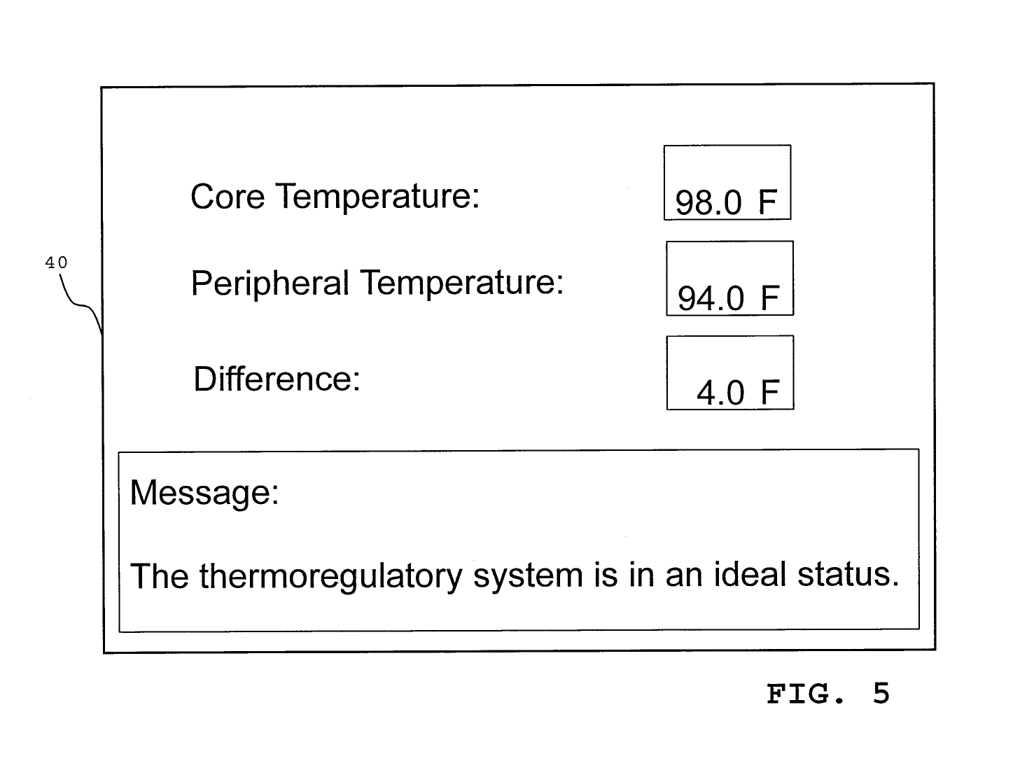

At S408, the processor/CPU 214 determines if the difference (ΔT) is above, within, or below the 1^{st} predetermined range (R1) changed at S406 according to Ta. Then, at S410, the processor/CPU 214 controls the user interface 40 to output 1^{st} alert according to the determination at S408. For example, when the difference (ΔT) is above the predetermined range R1, the message, such as, “The thermoregulatory system is in a low status” is displayed on the user interface 40 (e.g., LCD display) or such a message is read by the user interface 40 (e.g., speaker). In another example, when the difference (ΔT) is below the predetermined range R1, the message, such as, “The thermoregulatory system is in a high status” is displayed on the user interface 40 (e.g., LCD display) or such a message is read by the user interface 40 (e.g., speaker). In yet another example, when the difference (ΔT) is within the predetermined range R1, the message, such as, “The thermoregulatory system is in an ideal status” is displayed on the user interface 40 (e.g., LCD display) or such a message is read by the user interface 40 (e.g., speaker). FIG. 5 is an exemplary user interface of the human body thermal measurement device according to one embodiment where thermoregulatory system is determined to be in an ideal status. In this example, a core temperature, a peripheral temperature, a calculated difference and a message according to the determination at S408 is displayed on the user interface 40 (e.g., LCD display) or the message may be read by the user interface 40 (e.g., speaker). In another example, according to the determination at S408, the user interface 40 (e.g., LED lamp), as an indicator, may emit different colors of light (e.g., when thermoregulatory system is in a low status: red, high status: blue, ideal status: green). Then the step goes back to S404 and the cycle of S404, S406, S408 and S410 is repeated until the human body thermal measurement device 20 is turned off.

At S412, various types of errors are detected. For example, at S412, the processor/CPU 214 determines if at least one of the followings is applicable: (1) T1 is within a 2^{nd} predetermined range (R2), (2) T2 is within a 3^{rd} predetermined range (R3), (3) A change of T1 in time is within a 4^{th} predetermined range(R4), (4) A change of T2 in time is within a 5^{th} predetermined range(R5). By checking these items, situations such as at least either one of the 1^{st} sensor 202 or 2^{nd} sensor 204 is not firmly attached to the human body 10, or there is some trouble to the human body 10 can be detected.

Then at S414, if one of the items (1) to (4) is determined to be applicable, the processor/CPU 214 controls the user interface 40 (e.g., LCD display) to display alert message or controls the user interface 40 (e.g., speaker) to read out the alert message.

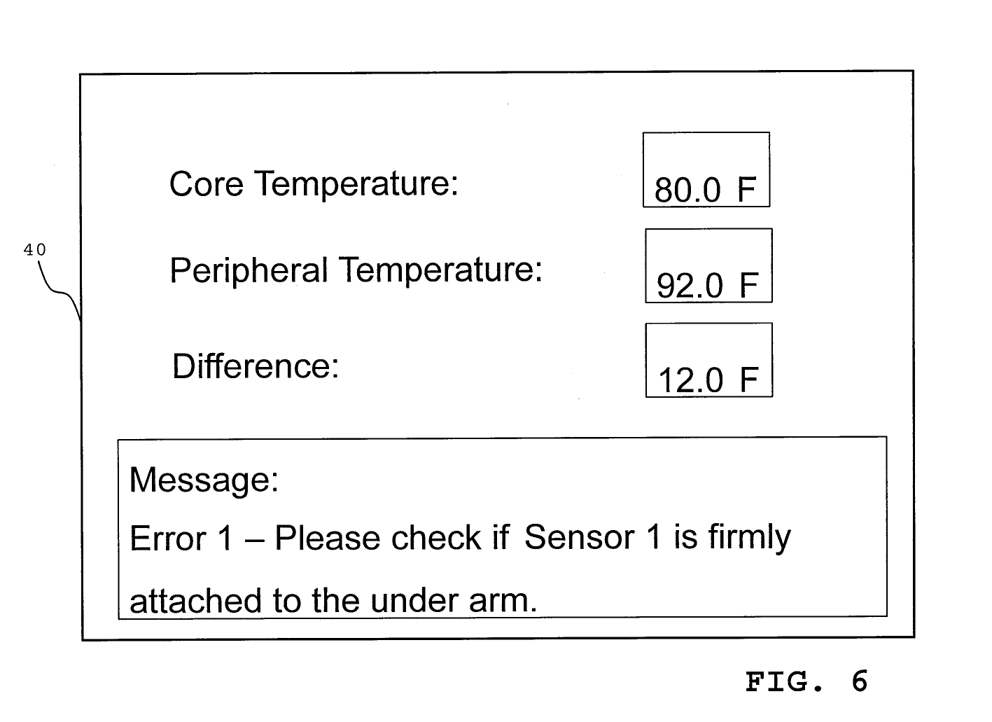

FIG. 6 is an exemplary user interface of the human body thermal measurement device according to another embodiment. In this example, a core temperature, a peripheral temperature, a calculated difference is displayed on the user interface 40. Then, for example, if core temperature T1 is determined to be below the 2^{nd} predetermined range (R2) (e.g., 92° F.), the processor/CPU 214 controls the user interface 40 (e.g., LCD display) to display alert message, such as “Error 1—Please check if Sensor 1 is firmly attached to the under arm.” or controls the user interface 40 (e.g., speaker) to read out the alert message.

In another example, either one of the 1^{st} predetermined range (R1), the 2^{nd} predetermined range (R2), the 3^{rd} predetermined range (R3), the 4^{th} predetermined range (R4), the 5^{th} predetermined range (R5), the 1^{st} predetermined temperature, or the 2^{nd} predetermined temperature can be changed from the user interface 40. Because each human body is different with each other, it is preferable these threshold ranges and values can be adjusted from the user interface 40 to compensate for the difference. For example, preset set of these threshold ranges and values according to physical characteristics (e.g., age, sex, height, weight) or pre-registered name may be stored in the memory 216, and based on the input from the user interface 40 on the physical characteristics or name, the processor/CPU 214 may automatically choose the suitable set of these threshold ranges and values.

FIG. 7 is an exemplary system for measuring human body temperature according to one embodiment. In this embodiment, the first human body thermal measurement device 20-1 and the second human body thermal measurement device 20-2 are connected to a server 60 via internet. A mobile device 70 is also connected to the server via internet, and the first human body thermal measurement device 20-1, the second human body thermal measurement device 20-2, the mobile device 70 and the server 60 can exchange data and communicate with each other through the internet. For example, a user may preregister a mobile terminal device 70 from the user interface 40, and when 1^{st} alert and/or 2^{nd} alert is output, the alert is automatically transmitted to the mobile terminal device 70 from the transmitter/receiver 222. This way, a user can notify the preregistered mobile terminal when some problem happens to the human body 10.

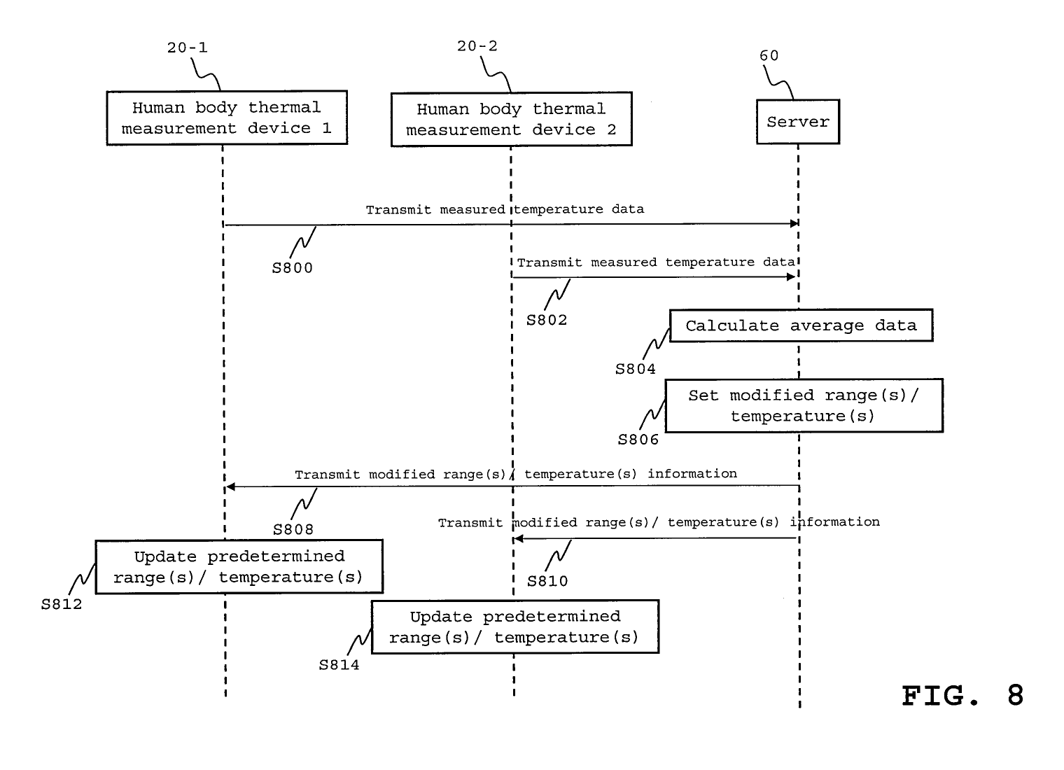

FIG. 8 is an exemplary flow chart of a system for measuring human body temperature according to one embodiment. In this example, at S800 and S802, both the first human body thermal measurement device 20-1 and the second human body thermal measurement device 20-2 transmit data on measured core temperature, peripheral temperature, and ambient temperature to the server 60. The first human body thermal measurement device 20-1 and the second human body thermal measurement device 20-2 may also transmit physical characteristics (e.g., age, sex, height, weight) of the human body measure to the server 60. Then at S804, the server 60 calculates average data for both core temperature and peripheral temperature according to ambient temperature. The average data may be sorted according to the physical characteristics of the human body. At S806, the server set modified range(s) and/or temperature(s) (e.g., the 1^{st} predetermined range (R1), the 2^{nd} predetermined range (R2), the 3^{rd} predetermined range (R3), the 4^{th} predetermined range (R4), the 5^{th} predetermined range (R5), the 1^{st} predetermined temperature, and/or the 2^{nd} predetermined temperature) according to the calculation at S804. At S808 and S810, the modified range(s) and/or temperature(s) information is transmitted to the first human body thermal measurement device 20-1 and the second human body thermal measurement device 20-2. Then, at S812 and S814, the first human body thermal measurement device 20-1 and the second human body thermal measurement device 20-2 update predetermined range(s) and/or temperature(s) information stored in the memory. This way, accuracy of predetermined range(s) and/or temperature(s) information is improved in time by sharing measurement data between devices. Therefore, by inputting physical characteristics (e.g., age, sex, height, weight) of the human body to be measured from the user interface 40 before measurement, a user can set suitable predetermined ranges and temperatures for the physical characteristics based on the past measurements.

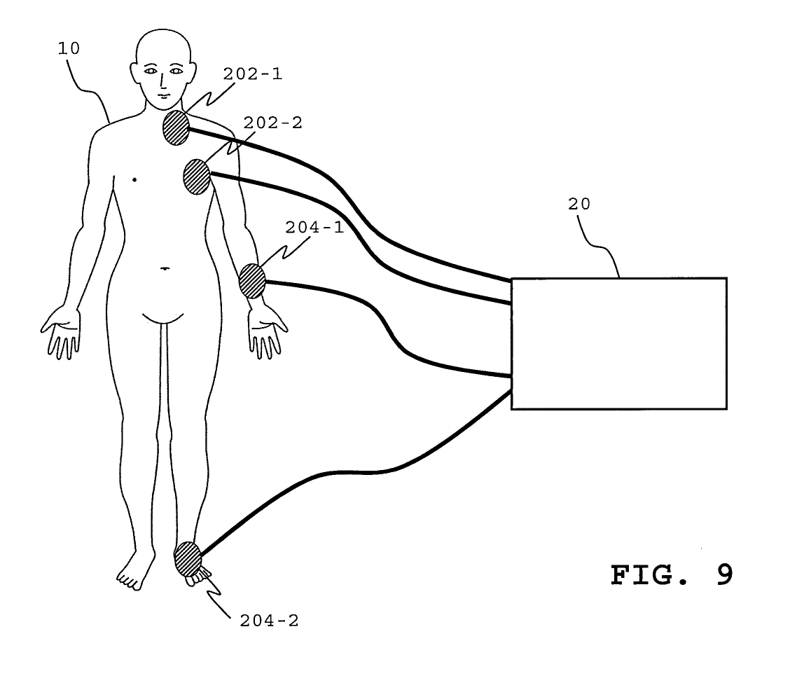

FIG. 9 is an exemplary system for measuring human body temperature according to another embodiment. In this embodiment, the human body thermal measurement device 20 includes plurality of 1^{st} sensors 202-1 and 202-2 that measure core temperatures of the human body 10, and plurality of 2^{nd} sensors 204-1 and 204-2 that measure the peripheral temperatures of the human body 10. For example, the processor/CPU 214 may calculate average of the data obtained by 1^{st} sensors 202-1 and 202-2, and/or 2^{nd} sensors 204-1 and 204-2. In another example, the processor/CPU 214 may eliminate either one of data obtained by 1^{St} sensors 202-1 and 202-2, and/or 2^{nd} sensors 204-1 and 204-2 that seems to be an error based on the calculation at S412 of FIG. 4, and use only rest of the data that seems to be reliable. This way, the reliability of measurement is improved.

Any processes, descriptions or blocks in flow charts should be understood as representing modules, segments, portions of code which include one or more executable instructions for implementing specific logical functions or steps in the process, and alternate implementations are included within the scope of the exemplary embodiment of the present system in which functions may be executed out of order from that shown or discussed, including substantially concurrently or in reverse order, depending upon the functionality involved, as would be understood by those skilled in the art. Further, it is understood that any of these processes may be implemented as computer-readable instructions stored on computer-readable media for execution by a processor.

Obviously, numerous modifications and variations of the present system are possible in light of the above teachings. It is therefore to be understood that within the scope of the appended claims, the system may be practiced otherwise than as specifically described herein.

1. A human body thermal measurement device comprising:
a first sensor configured to measure a first temperature of a core part of the human body;
a second sensor configured to measure a second temperature of a peripheral part of the human body;
a third sensor configured to measure an ambient temperature surrounding the human body;
a memory configured to store information on the first temperature, the second temperature and the ambient temperature;
circuitry configured to
calculate a difference between the first temperature and the second temperature;
change a first predetermined range according to the ambient temperature;
determine if the difference is within the first predetermined range or not;
determine if the first temperature is within a second predetermined range or not;
determine if the second temperature is within a third predetermined range or not;
determine if a change of the first temperature in time is within a fourth predetermine range or not; and
determine if a change of the second temperature in time is within a fifth predetermined range or not; and
a user interface configured to
output first alert when the difference is determined to be not within the first predetermined range by the circuitry; and
output second alert when either the first temperature is determined to be not within the second predetermined range, the second temperature is determined to be not within the third predetermined range, the change of the first temperature in time is determined to be not within the fourth predetermined range, or the change of the second temperature in time is determined to be not within the fifth predetermined range by the circuitry.

2. The human body thermal measurement device according to claim 1, wherein the circuitry changes the first predetermined range to a range with smaller value when the ambient temperature is higher than a first predetermined temperature, and/or changes the first predetermined range to a range with larger value when the ambient temperature is lower than a second predetermined temperature.

3. The human body thermal measurement device according to claim 2, further comprising a fourth sensor to detect if either one of the first sensor or the second sensor contacts with the human body.

4. The human body thermal measurement device according to claim 3, wherein the fourth sensor is a thermocouple sensor, a thermistor sensor, a resistance sensor or a galvanic skin response sensor.

5. The human body thermal measurement device according to claim 4, wherein either one of first predetermined range, second predetermined range, third predetermined range, fourth predetermined range, fifth predetermined range, the first predetermined temperature, or the second predetermined temperature can be changed from the user interface.

6. The human body thermal measurement device according to claim 5, wherein the circuitry determines if the difference is either above the first predetermined range, within the first predetermined range, or below the first predetermined range, and the circuitry controls the user interface to change the first alert according to the determination.

7. The human body thermal measurement device according to claim 6, wherein the user interface includes a display displaying a message, an indicator emitting plurality of colors of light, or a speaker outputting voice.

8. The human body thermal measurement device according to claim 7, wherein the first sensor and the second sensor are implemented in clothes.

9. A method for measuring human body temperature, the method comprising the steps of:
measuring a first temperature of a core part of the human body by a first sensor;
measuring a second temperature of a peripheral part of the human body by a second sensor;
measuring an ambient temperature surrounding the human body by a third sensor;
storing information on the first temperature, the second temperature and the ambient temperature;
calculating a difference between the first temperature and the second temperature by circuitry;
changing a first predetermined range according to the ambient temperature by the circuitry;
determining if the difference is within the first predetermined range or not by the circuitry;
determining if the first temperature is within a second predetermined range or not by the circuitry;
determining if the second temperature is within a third predetermined range or not by the circuitry;
determining if a change of the first temperature in time is within a fourth predetermine range or not by the circuitry;
determining if a change of the second temperature in time is within a fifth predetermined range or not by the circuitry;
outputting first alert when the difference is determined to be not within the first predetermined range by the circuitry; and
outputting second alert when either the first temperature is determined to be not within the second predetermined range, the second temperature is determined to be not within the third predetermined range, the change of the first temperature in time is determined to be not within the fourth predetermined range, or the change of the second temperature in time is determined to be not within the fifth predetermined range by the circuitry.

10. The method for measuring human body temperature according to claim 9, wherein the first predetermined range is changed to a range with smaller value by the circuitry when the ambient temperature is higher than a first predetermined temperature, and/or the first predetermined range is changed to a range with larger value by the circuitry when the ambient temperature is lower than a second predetermined temperature.

11. The method for measuring human body temperature according to claim 10, further comprising a step of detecting if either one of the first sensor or the second sensor contacts with the human body by a thermocouple sensor, a thermistor sensor, a resistance sensor or a galvanic skin response sensor.

12. The method for measuring human body temperature according to claim 11, further comprising the step of changing either one of first predetermined range, second predetermined range, third predetermined range, fourth predetermined range, fifth predetermined range, the first predetermined temperature, or the second predetermined temperature.

13. The method for measuring human body temperature according to claim 12, further comprising the step of determining by the circuitry if the difference is either above the first predetermined range, within the first predetermined range, or below the first predetermined range, and changing the first alert according to the determination.

14. The method for measuring human body temperature according to claim 13, wherein the step of outputting the first alert and outputting the second alert is carried out by displaying a message, emitting plurality of colors of light, or outputting voice.

15. A non-transitory computer readable storage medium including executable instructions, which when executed by a computer cause a computer to execute a method for use in a human body thermal measurement device, the method comprising the steps of:
measuring a first temperature of a core part of the human body by a first sensor;
measuring a second temperature of a peripheral part of the human body by a second sensor;
measuring an ambient temperature surrounding the human body by a third sensor;
storing information on the first temperature, the second temperature and the ambient temperature;
calculating a difference between the first temperature and the second temperature by circuitry;
changing a first predetermined range according to the ambient temperature by the circuitry;
determining if the difference is within the first predetermined range or not by the circuitry;
determining if the first temperature is within a second predetermined range or not by the circuitry;
determining if the second temperature is within a third predetermined range or not by the circuitry;
determining if a change of the first temperature in time is within a fourth predetermine range or not by the circuitry;
determining if a change of the second temperature in time is within a fifth predetermined range or not by the circuitry;
outputting first alert when the difference is determined to be not within the first predetermined range by the circuitry; and
outputting second alert when either the first temperature is determined to be not within the second predetermined range, the second temperature is determined to be not within the third predetermined range, the change of the first temperature in time is determined to be not within the fourth predetermined range, or the change of the second temperature in time is determined to be not within the fifth predetermined range by the circuitry.

16. The non-transitory computer readable storage medium according to claim 15, wherein the first predetermined range is changed to a range with smaller value by the circuitry when the ambient temperature is higher than a first predetermined temperature, and/or the first predetermined range is changed to a range with larger value by the circuitry when the ambient temperature is lower than a second predetermined temperature.

17. The non-transitory computer readable storage medium according to claim 16, further comprising a step of detecting if either one of the first sensor or the second sensor contacts with the human body by a thermocouple sensor, a thermistor sensor, a resistance sensor or a galvanic skin response sensor.

18. The non-transitory computer readable storage medium according to claim 17, further comprising the step of changing either one of first predetermined range, second predetermined range, third predetermined range, fourth predetermined range, fifth predetermined range or the predetermined temperature.

19. The non-transitory computer readable storage medium according to claim 18, further comprising the step of determining by the circuitry if the difference is either above the first predetermined range, within the first predetermined range, or below the first predetermined range, and changing the first alert according to the determination.

20. The non-transitory computer readable storage medium according to claim 19, wherein the step of outputting the first alert and outputting the second alert is carried out by displaying a message, emitting plurality of colors of light, or outputting voice.
